(19) United States
(12) Patent Application Publication
(10) Pub. No.: US 2022/0354773 A1
(43) Pub. Date: Nov. 10, 2022

(54) ROSEBUSH EXTRACT

(71) Applicant: L'OREAL, Paris (FR)

(72) Inventors: Dominique FAGOT, Aulnay-Sous-Bois (FR); Geraldine DEJEAN, Chevilly-Larue (FR)

(73) Assignee: L'OREAL, Paris (FR)

(21) Appl. No.: 17/622,587

(22) PCT Filed: Jun. 18, 2020
(86) PCT No.: PCT/EP2020/067026

§ 371 Date: Dec. 23, 2021

(30) Foreign Application Priority Data
Jun. 28, 2019 (FR) FR1907145

(51) Int. Cl. A61K 8/9789; A61Q 19/08

(52) U.S. Cl. CPC A61K 8/9789; A61Q 19/08; A61K 2800/805

(57) ABSTRACT

The present invention relates to a rosebush extract, characterized in that said rosebush is a hybrid obtained by crossing the varieties Meichibon×Delgramaue. In particular, the present invention relates to a cosmetic use and a cosmetic process for caring for keratin materials by using said rosebush extract.

### TECHNICAL FIELD

The present invention relates to the field of active agents dedicated to caring for keratin materials, such as the skin or skin integuments, and in particular for acting towards skin aging. The applications relate mainly to the field of cosmetics.

Human skin consists of several compartments, three of which cover the whole of the body, namely a superficial compartment, which is the epidermis, the dermis and a deep compartment, which is the hypodermis.

The hypodermis consists essentially of a type of cells that are specialized in the accumulation and storage of fats, the adipocytes.

The dermis is a connective tissue, consisting of collagen fibers and elastic fibers and also glycosaminoglycans, proteoglycans and fibroblasts. Its architecture results from the arrangement and interactions between the extracellular matrix constituents and the fibroblasts which are responsible for the synthesis and degradation thereof. This extracellular matrix is predominantly composed of elastin and of collagen. Collagen is a fibrous protein present in the extracellular medium of all connective tissues. Among the 20 identified types of collagen, collagens I and III are the major components of the dermis. They are secreted into the extracellular matrix by fibroblasts in the form of procollagens, consisting of three α-polypeptide chains forming a helical structure.

The dermo-epidermal junction (DEJ) or basal membrane consists of leaflets of extracellular matrix separating cells of different origin: keratinocytes and fibroblasts. The main constituents of this DEJ are collagen IV, a non-fibrillar protein forming a two-dimensional network and proteoglycans such as laminin, nidogen and perlecan. Finally, collagen VII molecules secreted by the keratinocytes and the fibroblasts form anchoring fibers which provide cohesiveness between the basal membrane of the epidermis and the dermis. Finally, the epidermis consists mainly of keratinocytes, but also of other cells, in particular melanocytes. These cells are located in a basal membrane which separates them from the dermis. Melanocytes are specialized dendritic cells whose function is to synthesize melanin. Schematically, three types of epidermal cells participate in this system: keratinocytes, melanocytes and certain resident lymphocytes. These cells, which are only found in the skin, play an essential role in cicatrization and in the re-epithelialization phenomena. Re-epithelialization may thus be conceptually defined as the result of three functions of the keratinocytes: migration, proliferation and differentiation.

Skin aging results from two distinct and independent processes which involve intrinsic or extrinsic factors. Intrinsic or chrono-biological aging corresponds to normal or physiological aging which is age-related.

With time, and notably in the course of chronological and/or photo-induced aging, the skin undergoes numerous modifications and degradations which are reflected, at the tissue level, by a disorganisation of the architecture of the epidermis, the dermo-epidermal junction, the dermis, and also of the blood supply and innervation systems, and a slowing down or deregulation of various cell metabolisms, such as those involved in the equilibrium of the barrier function or those involved in melanogenesis. At the cellular level, aging is reflected by impairment of the physiology or metabolism of the main cell types, such as the fibroblasts of the dermis, the keratinocytes of the epidermis, and also the melanocytes.

Intrinsic aging is notably reflected by a slowing down of the renewal of epidermal cells and the appearance of wrinkles or fine lines. At the level of the dermis, the biosynthesis of macromolecules such as collagen decreases with age, changing the mechanical properties of the dermis, whence arises slackening of the skin, which is one of the clinical signs of aging. Extrinsic aging corresponds to aging generally caused by the environment and corresponds more particularly to photoaging due to exposure to sunlight. Photo-induced skin aging, i.e. caused by exposure to sunlight, is also known as photoaging or heliodermia.

Photoaging is the result, at the level of the dermis, of degradation of the collagen fibers, the consequence of which is notably clinical impairments such as thick wrinkles and the formation of a slackened and leathery skin. Skin aging is thus accelerated by chronic exposure to UV light.

Thus, healthy skin is capable of defending itself against external stresses notably by means of its barrier and antimicrobial defence properties, and also its re-epithelialization properties. In the long term, these stresses may be reflected by a depressor effect on the barrier properties of the skin.

These stresses may also affect the re-epithelialization properties and impair the processes of epidermal renewal and of cicatrization, notably those causing the signs of skin aging. From a cosmetic point of view, by promoting re-epithelialization, and notably keratinocyte migration, it is thus possible to prevent and/or treat the signs associated with skin aging.

### PRIOR ART

Various compounds have been proposed for caring for keratin materials, notably in the cosmetic field.

By way of example, mention may be made of FR 2 890 311, which teaches a cosmetic use of a plant extract from the genus Rosa, for preventing or reducing the adhesion of microorganisms to the surface of the skin and/or mucous membranes.

FR 2 985 423 teaches the cosmetic use of de-differentiated plant cells from Rosa sp. for the esthetic care of the skin and the hair.

Deshayes et al. (“A 3D in vitro model of the re-epithelialization phase in the wound-healing process”; Experimental Dermatology; Vol. 27, Issue 5, 2017) moreover reports an in vitro model of re-epithelialization, in which punicic acid, ellagic acid and ascorbic acid are identified as pro-cicatrizing active agents.

### DISCLOSURE OF THE INVENTION

However, there is an ongoing need for new active agents that are useful for caring for keratin materials. In particular, there is still a need for new active agents that are natural and that have a positive effect on keratin materials.

There is still a need for new active agents that are useful for reinforcing the re-epithelialization, renewal and cicatrization properties of the epidermis.

There is still a need for new active agents that are suitable for preventing and/or treating the signs of aging of keratin materials.

There is still a need for new useful active agents that are capable of reinforcing the barrier properties of the skin.

The aim of the present invention is to satisfy these needs.

### SUMMARY OF THE INVENTION

According to a first subject, the invention relates to a rosebush extract, characterized in that said rosebush is a hybrid obtained by crossing the varieties Meichibon×Delgramaue.

In particular, said rosebush extract may be obtained from flowers, flowering tops and/or leaves of said rosebush.

In particular, said rosebush extract may be obtained by extraction with supercritical CO₂ of an alcoholic mixture of all or part of said rosebush.

In particular, said rosebush extract may be characterized in that said alcoholic mixture is obtained after infusion of all or part of said rosebush in at least one bath comprising an alcoholic solvent, at a temperature of less than 50° C., in order to obtain an alcoholic mixture.

According to a second subject, the invention relates to a composition comprising a rosebush extract as defined previously.

According to a third subject, the invention relates to a cosmetic use of an extract of a hybrid rosebush obtained by crossing the varieties Meichibon×Delgramaue, or of a composition comprising said extract, for caring for keratin materials.

In particular, said cosmetic use may be for the purpose of treating and/or preventing cosmetic signs chosen from wrinkles, fine lines, wizened skin, loss of elasticity and/or tonicity and/or density of the skin, impairment of the radiance of the skin complexion, the papery appearance of the skin, slackening of the skin, and the wizened appearance of the skin.

According to a fourth subject, the invention relates to a cosmetic process for caring for keratin materials, comprising at least one step consisting in administering to an individual in need thereof, as an active agent, at least one rosebush extract, characterized in that said rosebush is a hybrid obtained by crossing the varieties Meichibon×Delgramaue.

### BRIEF DESCRIPTION OF THE DRAWINGS

FIG. 1 represents a general protocol for obtaining a rose extract par extraction with supercritical CO₂ of a floral infusion. The masses are indicative values, which may be subject to variation.

### DETAILED DESCRIPTION

Surprisingly, the inventors have identified, in a keratinocyte model in culture, the advantageous properties of extracts from particular hybrid rosebushes, obtained by crossing the varieties Meichibon×Delgramaue.

The variety name Meichibon refers to a rosebush belonging to the Rosaceae family, from the genus Rosa. It is a hybrid tea rose, also commercially referred to as Tchaikovski®, or Tchaikovski® Meichibon, or Meilland rosebush.

The variety name Delgramaue refers to a rosebush belonging to the Rosaceae family, from the genus Rosa, from the species Floribunda, also commercially referred to as “rose synactif by Shisheido®” (Delbard), or “La Rose du Petit Prince”.

Such a hybrid rosebush may present abundant white double leaves, which may show some pink colored tips, i.e. an average of five flowers per stem, and also a fragrance presenting various notes, including (i) a top note of grapefruit and citrus rose, (ii) a heart note of apricot and litchi and (iii) a green base note. It may reach, on average, a height of about 70 to 80 cm, and a width of about 40 to 50 cm, with branches having a diameter of between about 8 and 10 mm.

In particular, such a hybrid rosebush may be obtained by hybridization of a “male” variety of the variety name Delgramaue, and of a “female” variety of the variety name Meichibon. In particular, such a hybrid rosebush may be obtained by pollination, i.e. application of a pollen from the stamens of a “male” flower, and in particular of a flower belonging to the variety name Delgramaue, on the pistil of a “female” flower, and in particular of a flower belonging to the variety name Meichibon.

This hybrid rosebush may notably be distinguished from the varieties Meichibon and Delgramaue, defined previously, by virtue of a combination of the following features:
the number of petals generally differs from the Meichibon variety, in that this type of rosebush has flowers with a greater number of petals, also of a bigger size, and with a stronger fragrance with, as stated previously, a characteristic note of grapefruit;
the color of the petals generally differs from the Delgramaue variety, in that their color is generally white, whereas the Delgramaue variety has petals of a lilac color, it is more vigorous and more resistant toward the disease known as “black spot disease of roses”. Thus, the inventors identified the pro-migration and re-epithelialization capacities of said hybrid rosebush extract in an in vitro model of keratinocyte culture described in Deshayes et al. (“A 3D in vitro model of the re-epithelialization phase in the wound-healing process”; Experimental Dermatology; Vol. 27, Issue 5, 2017).

These pro-migration and re-epithelialization capacities may advantageously be implemented for cosmetic or non-cosmetic applications, or for the preparation of compositions, notably cosmetic compositions, for treating and/or preventing signs associated with a migration or re-epithelization defect.

In particular, it was shown that a supercritical CO₂ extract of said hybrid rosebush induces stimulation of the migration of normal human keratinocytes in a system which can mimic an area of an artificial and homogeneous wound. The results obtained with the extract have the same amplitude as those obtained with the positive control (EGF). A 50% acceleration of migration relative to the positive control is thus observed, from the first hours of migration. A supercritical CO₂ extract generally refers to an extract obtained by a process using CO₂ gas in a “supercritical” state, i.e. at a high pressure level (generally greater than 50 bar, or even greater than 70 bar), and at a low temperature (generally greater than 30° C. and lower than 50° C.).

According to one embodiment, the extraction is performed in the presence of a CO₂ gas in the supercritical state, i.e. at a temperature of at least 31.1° C. and at a pressure of at least 74.5 bar.

Said supercritical CO₂ extract may notably be obtained according to a protocol described in WO 2012/085366 and detailed hereinbelow.

An extract of a hybrid rosebush, obtained by crossing the varieties Meichibon×Delgramaue, may thus advantageously be used for caring for keratin materials, in particular the skin and its integuments, and most particularly for treating and/or preventing the signs of skin aging, such as wrinkles, fine lines, wizened skin, loss of elasticity and/or tonicity and/or density of the skin, impairment of the radiance of the skin complexion, the papery appearance of the skin, slackening of the skin, the wizened appearance of the skin.

The term “keratin materials” is intended to denote the skin and its integuments, notably the scalp, the hair follicles and keratin fibers, notably head hair, the eyebrows, the eyelashes, beard and moustache hair and pubic hair.

The term “skin” means all of the skin of the body, including the scalp, mucous membranes, semimucous membranes, and the skin integuments.

The term “skin integuments” means bodily hair, the eyelashes, head hair and the nails. More particularly, in the present invention, head hair, the skin of the neckline, of the neck and of the face, the eyelashes and the eyebrows are considered.

The term “preventing” also means “reducing the probability of occurrence or of recurrence of a phenomenon”.

The term “signs of skin aging” refers to all the modifications of the external appearance of the skin due to aging, whether it be chronobiological and/or photo-induced, for instance wrinkles and fine lines, wizened skin, lack of elasticity and/or tonicity of the skin, thinning of the dermis and/or degradation of the collagen fibers, which result in the skin appearing flaccid and wrinkled. It is also intended to refer to all the internal modifications of the skin which are not systematically reflected by a modified external appearance, for instance all the internal degradations of the skin, and more particularly the degradation of elastin fibers, or elastic fibers.

### Extracts, Compositions and Preparation Processes

According to a main embodiment, the invention relates to a rosebush extract, characterized in that said rosebush is a hybrid obtained by crossing the varieties Meichibon×Delgramaue. Rose extracts according to the invention may be obtained from plant material derived from whole plants or from plant parts, such as the leaves, stems, flowers, flowering tops, petals, sepals or roots cultivated in vivo or in vitro.

The term “in vivo culture” means any culture of standard type, i.e. in soil in the open air or in a greenhouse, or alternatively out of the soil.

The term “in vitro culture” means all the techniques known to those skilled in the art which make it possible to artificially obtain a plant or a plant part. The imposed selection pressure makes it possible to obtain a plant material which is standardized and available all year round, contrary to plants cultivated in vivo.

In particular, said rosebush extract may be obtained from flowers, flowering tops, and/or leaves.

According to a second embodiment, the invention relates to a cosmetic or pharmaceutical composition, and preferably a cosmetic composition, comprising an extract of said rosebush, characterized in that said rosebush is a hybrid obtained by crossing the varieties Meichibon×Delgramaue.

### Formulations

Extracts according to the invention may be formulated in any cosmetic composition, notably for application to the skin, the nails or mucous membranes (buccal, jugal, gingival, genital, connective). Depending on the retained administration method, a composition of the invention may be in any of the presentation forms normally used.

A composition according to the invention comprises a physiologically acceptable medium. The term “physiologically acceptable medium” means a medium that is compatible with keratin materials, in particular the skin. According to one embodiment, an extract according to the invention may be administered via a topical route.

According to one embodiment, the rosebush extract according to the invention may be present and/or administered in a composition comprising at least 0.0001% by weight, relative to the total weight of the composition.

According to one embodiment, the rosebush extract according to the invention may be present and/or administered in a composition comprising at least 0.001% by weight, relative to the total weight of the composition.

According to one embodiment, the rosebush extract according to the invention may be present and/or administered in a composition comprising not more than 0.1% by weight, relative to the total weight of the composition.

According to one embodiment, the rosebush extract according to the invention may be present and/or administered in a composition comprising not more than 1% by weight, relative to the total weight of the composition.

According to one embodiment, the rosebush extract according to the invention may be present and/or administered in a composition comprising at least 0.0001% by weight, and not more than 1% by weight, relative to the total weight of the composition.

According to one embodiment, the rosebush extract according to the invention may be present and/or administered in a composition comprising at least 0.0001% by weight, and not more than 0.1% by weight, relative to the total weight of the composition.

Advantageously, the extracts according to the invention may be formulated or dissolved in water or a water-soluble organic solvent, or a mixture thereof.

A water-soluble organic solvent that is suitable for use in the invention may be chosen from lower monoalcohols including from 2 to 8 atoms, and C₂ to C₈, preferably C₃ to C₆, hydrocarbon-based compounds comprising from 2 to 6 hydroxyl groups, preferably from 3 to 5 hydroxyl groups, and mixtures thereof.

Among the water-soluble organic solvents that are suitable for use in the invention, mention may notably be made of glycols containing from 2 to 8 carbon atoms, such as ethylene glycol, propylene glycol or 1,3-propanediol, 1,3-butylene glycol, dipropylene glycol, glycerol, sorbitol, and mixtures thereof. Preferably, propylene glycol or 1,3-propanediol is most particularly suitable for use in the invention.

Among the lower monoalcohols, mention may in particular be made of those including from 2 à 6 carbon atoms, such as ethanol, isopropanol, propanol or butanol.

In a preferred embodiment, the water-soluble organic solvent is ethanol.

A water-soluble organic solvent may constitute from 20% to 100% by weight of the composition containing it, preferably from 30% to 90%, preferably from 40% to 80%, and more preferably from 50% to 70% by weight of the composition containing it.

A water that is suitable for use in the invention may be a spring and/or mineral water, notably chosen from Vittel water, waters from the Vichy basin and La Roche Posay water. A water that is suitable for use in the invention may also be a floral water, such as rose water.

Water may constitute may constitute from 20% to 100% by weight of the composition containing it, preferably from 30% to 90%, preferably from 40% to 80%, and more preferably from 50% to 70% by weight of the composition containing it. Advantageously, water constitutes up to 50% by weight of the composition containing it.

For topical application to keratin materials, and notably the skin or its integuments, a composition may notably be in the form of an aqueous or oily solution or of a dispersion of the lotion or serum type, of emulsions of liquid or semi-liquid consistency of the milk type, obtained by dispersing a fatty phase in an aqueous phase (0/W), or conversely (W/O), or of suspensions or emulsions of soft consistency, of the aqueous or anhydrous gel or cream type, or else of microcapsules or microparticles, or of vesicular dispersions of ionic and/or nonionic type. These compositions are prepared according to the usual methods.

These compositions may constitute cleansing, protective, treating or care creams for the face, the hands, the feet, the major anatomical folds or the body (for example day creams, night creams, makeup creams, makeup-removing creams, foundation creams or antisun creams), fluid foundations, makeup compositions such as makeup-removing milks, protective or care body milks, antisun milks, skincare lotions, gels or foams, for instance cleansing lotions, antisun lotions, artificial tanning lotions, bath compositions, deodorant compositions comprising a bactericidal agent, aftershave gels or lotions, or hair-removing creams. These compositions may also consist of solid preparations constituting soaps or cleansing bars or may be packaged in the form of an aerosol composition also comprising a pressurized propellant.

A composition for making up keratin materials, such as the eyelashes or the eyebrows, may be chosen in particular from: a mascara, an eyeliner, a lipstick, a liquid foundation or a powder.

According to one embodiment, a composition according to the invention may comprise:
at least one oil, such as a volatile oil, and notably a volatile hydrocarbon-based oil; and/or
at least one fatty substance, such as a fatty substance which is solid at 25° C.

The amounts of the various constituents in the compositions according to the invention are those conventionally used in the fields under consideration.

When a composition is an emulsion, the proportion of the fatty phase may range from 5% to 80% by weight and preferably from 5% to 50% by weight relative to the total weight of the composition. The oils, waxes, emulsifiers and coemulsifiers used in a composition in emulsion form are chosen from those conventionally used in the cosmetics field. The emulsifier and the coemulsifier may be present, in a composition, in a proportion ranging from 0.3% to 30% by weight and preferably from 0.5% to 20% by weight relative to the total weight of the composition.

When a composition is an oily solution or gel, the fatty phase may represent more than 90% of the total weight of the composition.

In a known manner, a cosmetic composition of the invention may also contain adjuvants that are customary in the cosmetics field, such as hydrophilic or lipophilic gelling agents, hydrophilic or lipophilic additives, preserving agents, antioxidants, solvents, fragrances, fillers, screening agents, odor absorbers and dyestuffs. The amounts of these various adjuvants are those conventionally used in the cosmetics field, and are, for example, from 0.01% to 10% of the total weight of the composition. Depending on their nature, these adjuvants may be introduced into the fatty phase, into the aqueous phase and/or into lipid spherules.

As oils or waxes that may be used in the invention, mention may be made of mineral oils (liquid petroleum jelly), plant oils (liquid fraction of shea butter, sunflower oil), animal oils (perhydrosqualene), synthetic oils (purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils.

As emulsifiers that may be used in the invention, mention may be made, for example, of glyceryl stearate, polysorbate 60, and the PEG-6/PEG-32/glycol stearate mixture sold under the name Tefose® 63 by the company Gattefosse.

As solvents that may be used in the invention, mention may be made of lower alcohols, notably ethanol, isopropanol and propylene glycol.

As hydrophilic gelling agents that may be used in the invention, mention may be made of carboxyvinyl polymers (carbomer), acrylic copolymers such as acrylate/alkyl acrylate copolymers, polyacrylamides, polysaccharides such as hydroxypropylcellulose, natural gums, preferably xanthan gum, and clays, and, as lipophilic gelling agents, mention may be made of modified clays such as bentones, metal salts of fatty acids, for instance aluminum stearates, and hydrophobic silica, ethylcellulose and polyethylene.

### Preparation Processes

A hybrid rosebush extract according to the invention, obtained by crossing the varieties Meichibon×Delgramaue, may be obtained by any known means.

For example, an extract according to the invention may be obtained by extraction with apolar volatile solvents derived from petrochemistry, such as hexane, isohexane, cyclohexane, benzene, petroleum ether, propane or butane. The water from the plants is then allowed to settle, and the solvent containing the perfume is concentrated under vacuum to yield the extracted perfume essence. An extract according to the invention may also be obtained by steam distillation or by hydrodistillation.

Said extract may be an alcoholic mixture obtained by infusing all or part of said rosebush in at least one bath comprising an alcoholic solvent.

The general process of extraction with supercritical CO₂ is known. In the supercritical state, i.e. at more than 74 bar (in particular more than 74.4 bar) and more than 31° C. (in particular more than 31.1° C.), CO₂ has very special properties and may be used as a natural extraction solvent. The obtained fluid is characterised by high diffusivity (of the order of that of gases) which gives it good ability for diffusion, and a high density which imparts a high capacity for transport and extraction.

In a preferred manner, a rosebush extract according to the invention is obtained by an extraction process with supercritical CO₂ of all or part of said rosebush, and notably according to any one of the variants described in WO 2012/085366, the contents of which are incorporated by reference in the present description.

According to this preferred embodiment, said rosebush extract may thus be obtained by extraction with supercritical CO₂ of an alcoholic mixture of all or part of said rosebush. Said alcoholic mixture may also be obtained by infusing all or part of said rosebush in at least one bath comprising an alcoholic solvent.

The extraction step with supercritical CO₂ according to the invention may be performed in static mode or in dynamic mode.

According to the invention, the CO₂ is preferentially used at a pressure of between 130 and 200 bar and at a temperature of between 35 and 55° C., even more preferentially at 150 bar and 45° C., in counter-current mode, and is particularly suitable for obtaining an extract of fresh flowers and/or leaves, which is clear, transparent and stable, mostly freed of sugars, coloring matter, and water, and having an alcohol titer of at least 75%.

Advantageously, the process according to the invention also comprises a step in which the extract obtained after extraction with supercritical CO₂ is concentrated as obtained, under vacuum with mild heating at a temperature of less than 60° C., or on a support such as a natural oil, shea butter, natural glycerol, or a natural fragrant molecule such as natural benzyl acetate, natural geraniol, or natural nerolidol.

As an example of an alcoholic solvent according to the invention, a natural alcohol chosen from methanol, ethanol, 1-propanol, 2-propanol, butanol, isobutanol, pentanol and isoamyl alcohol, preferentially ethanol, is used, which has a lower boiling point (except for methanol) and which is much less toxic than, notably, methanol. An alcoholic solvent may be an ethanolic solvent.

Most particularly, said alcoholic mixture may be obtained after infusion of flowers, flowering tops, and/or leaves in at least one bath comprising an alcoholic solvent, at a temperature of less than 50° C., in order to obtain an alcoholic or aqueous-alcoholic mixture, or even a fragranced alcoholic or aqueous-alcoholic mixture.

According to the invention, the flowers, flowering tops and/or leaves are preferentially infused in the alcoholic solvent at room temperature, i.e. a temperature of between 15 and 35° C.

An alcoholic mixture may thus be obtained by infusing all or part of said rosebush in at least one bath comprising an alcoholic solvent.

A rosebush extract according to the invention may notably comprise volatile compounds, and in particular at least one compound chosen from: cis-3-hexenol, trans-2-hexenol, C₆ alcohol, diethoxyethanol, methylheptenone, acetin or a related compound, cis-3-hexenyl acetate, hexyl acetate, phenylacetaldehyde, benzyl alcohol, linanol, phenylethyl alcohol, diacetin or a related compound, benzyl acetate, diethyl succinate, terpinen-4-ol, nerol, citronellol, geraniol, geranial, cistheaspirane, delta-elemene, citronellyl acetate, geranyl acetate, alpha-copaene, beta-elemene, coumarin, hydroxyedulane or isomer, β-caryophyllene, dihydro-β-ionin, dihydro-β-ionol, α-jumulene, γ-muurolene, germacrene D, α-cadinene, β-bisabolene, γ-cadinene, γ-eudesmol, β-eudesmol, α-cadinol, 4-oxo-dihydro-β-ionol, benzyl benzoate, ethyl myristate, C₁₉ alkene, C₁₉ alkane, palmitic acid, ethyl palmitate, C₂₀ alkane, C₂₁ alkane, linoleic acid, linolenic acid, ethyl linoleate, ethyl linolenate, ethyl stearate, tricosene, tricosane, dihydro-β-ionol ester.

In particular, a rosebush extract according to the invention may notably comprise a plurality of compounds chosen from: cis-3-hexenol, trans-2-hexenol, C₆ alcohol, diethoxyethanol, methylheptenone, acetin or a related compound, cis-3-hexenyl acetate, hexyl acetate, phenylacetaldehyde, benzyl alcohol, linanol, phenylethyl alcohol, diacetin or a related compound, benzyl acetate, diethyl succinate, terpinen-4-ol, nerol, citronellol, geraniol, geranial, cistheaspirane, delta-elemene, citronellyl acetate, geranyl acetate, alpha-copaene, beta-elemene, coumarin, hydroxyedulane or isomer, β-caryophyllene, dihydro-β-ionin, dihydro-β-ionol, α-jumulene, γ-muurolene, germacrene D, α-cadinene, β-bisabolene, γ-cadinene, γ-eudesmol, β-eudesmol, α-cadinol, 4-oxo-dihydro-β-ionol, benzyl benzoate, ethyl myristate, C₁₉ alkene, C₁₉ alkane, palmitic acid, ethyl palmitate, C₂₀ alkane, C₂₁ alkane, linoleic acid, linolenic acid, ethyl linoleate, ethyl linolenate, ethyl stearate, tricosene, tricosane, dihydro-β-ionol ester.

A rosebush extract according to the invention, notably obtained by extraction with supercritical CO₂, may, for example, comprise at least one compound chosen from: geraniol, geranial, nerol and citronellol.

According to one embodiment, a rosebush extract according to the invention may also comprise at least one compound chosen from: cis- or trans-theaspirane, dihydro-beta-ionone, dihydro-beta-ionol, 4-oxodihydronetaionol.

During infusion, the flowers, flowering tops and/or leaves are soaked in the alcoholic solvent and may be gently swirled.

Advantageously, the infusion is performed using solvent circulation in a closed circuit, i.e. the solvent is circulated on the flowers, flowering tops and/or leaves so as to create a movement in the extractor, notably without breaking the petals, and to avoid saturation areas of the solvent around the petals. The swirling thus provides solvent which is less saturated and which will in turn perform the extraction. Alternatively, infusions may be performed in several concomitant or successive baths, depending on the quantity of flowers and/or leaves to be treated.

It is possible, for example, to prepare a single bath, followed by rinsing using fresh extraction solvent, several baths with the same flowers and/or leaves, or even several runs of flowers and/or leaves in the same bath due to the low saturation of the ethanol, for a final weight/weight ratio of flowers-leaves/alcoholic solvent of from 1:1 to 1:10, preferentially from 1:1 to 1:3.

For example, advantageously several reruns of flowers and/or leaves may be performed in the same alcoholic bath in order to saturate it, for example up to five reruns, which makes it possible to concentrate the primary alcoholic extract. This proves to be more economical in terms of volumes to be transported and treated when the process for obtaining said extract involves an extraction step with supercritical CO₂.

Next, according to the process, the flowers, flowering tops and/or leaves are generally drained, avoiding excessive crushing, and the alcoholic mixture thus obtained is filtered so as to collect an alcoholic floral infusion suitable for keeping cool at a temperature from about 4 to 10° C. for one day to several months.

A process for obtaining a rosebush extract may thus comprise the following steps:

a) infusing all or part of a hybrid rosebush obtained by crossing the varieties Meichibon×Delgramaue, in at least one bath comprising an alcoholic solvent, in particular an ethanolic solvent, at a temperature of less than 50° C., so as to obtain an alcoholic mixture;
b) optionally filtering said alcoholic mixture so as to collect an alcoholic floral infusion; and
c) performing an extraction with supercritical CO₂ of said alcoholic mixture or of said alcoholic floral infusion so as to obtain said extract.

A process for obtaining a rosebush extract may comprise the following steps:

a) picking the flowers, flowering tops and/or leaves of a hybrid rosebush obtained by crossing the varieties Meichibon×Delgramaue;
b) infusing the flowers, flowering tops and/or leaves provided in step a), in at least one bath comprising an alcoholic solvent, in particular an ethanolic solvent, at a temperature of less than 50° C., so as to obtain an alcoholic mixture;
c) optionally filtering said alcoholic mixture so as to collect an alcoholic floral infusion; and
d) performing an extraction with supercritical CO₂ of said alcoholic mixture or of said alcoholic floral infusion so as to obtain said extract.

### Cosmetic or Therapeutic Indications

An extract of a hybrid rosebush, obtained by crossing the varieties Meichibon×Delgramaue, and according to the invention, promotes and reinforces the cicatrization and re-epithelialization phenomena.

An extract according to the invention thus makes it possible to increase the resistance of the skin barrier, notably by reinforcing the re-epithelialization phenomena.

The experimental data collected on an in vitro model of re-epithelialization show that said extract is an efficient active agent for improving the re-epithelialization and migration phenomena, for notably reducing, or even delaying or preventing, the accumulation of damaged epidermal cells and also improving epidermal regeneration.

All these effects have enabled the inventors to define a new active composition, the properties of which prove to be particularly advantageous and noteworthy for caring for keratin materials, notably regarding skin disorders associated with re-epithelialization, notably cicatrization disorders, or involving an age-related deficiency of the re-epithelialization process, in the skin or the hair follicles.

A deficiency of the re-epithelialization processes may be caused or aggravated by chrono-biological or photo-induced aging of the skin.

According to one of its main subjects, the invention thus relates to the cosmetic use of an extract of a hybrid rosebush obtained by crossing the varieties Meichibon×Delgramaue, or of a composition comprising said extract, for caring for keratin materials.

According to a particular embodiment, the signs considered are those which may be linked to aging, notably to skin aging.

In particular, the signs of skin aging targeted by the invention relate to all the modifications of the external appearance of the skin due to aging, whether it be chronological and/or photo-induced, for instance thinning of the epidermis and/or loss of firmness, elasticity, density and/or tonicity of the epidermis and/or the formation of wrinkles and fine lines.

Said extract may thus be implemented in the context of a cosmetic use for treating and/or preventing cosmetic signs chosen from wrinkles, fine lines, wizened skin, loss of elasticity and/or tonicity and/or density of the skin, impairment of the radiance of the skin complexion, the papery appearance of the skin, slackening of the skin, the wizened appearance of the skin.

According to another subject, the present invention relates to a cosmetic process for caring for keratin materials, comprising at least one step consisting in administering to an individual in need thereof, as an active agent, at least one rosebush extract, characterized in that said rosebush is a hybrid obtained by crossing the varieties Meichibon×Delgramaue.

According to yet another of its subjects, the present invention relates to a pharmaceutical or dermatological composition comprising said extract, for preventing and/or treating re-epithelialization disorders, notably cicatrization.

Alternatively, and according to another subject, the present invention relates to the use of said extract for the preparation of a composition for preventing and/or treating re-epithelialization disorders, notably cicatrization.

According to a preferred embodiment, an extract according to the invention may be administered via a topical route.

### EXAMPLE

### Example 1

### Production of a Supercritical CO₂ Extract of White Rose

A hybrid variety of rose is obtained by controlled crossing of two varieties: a maternal variety (Tchaikovsky® Meichibon) and a paternal variety (La Rose du Petit Prince/Rose Synactif by Shiseido® Delgramaue).

The maternal line is also known as its plant name: Meichibon. The paternal line is also known as its plant name: Delgramau.

A protocol implemented for obtaining a “supercritical CO₂” extract is the protocol described in patent application WO 2012/085366.

Briefly, this protocol consists in obtaining an extract from fresh and/or slightly withered rose flowers, flowering tops and/or leaves according to the invention, comprising the following steps, in which:

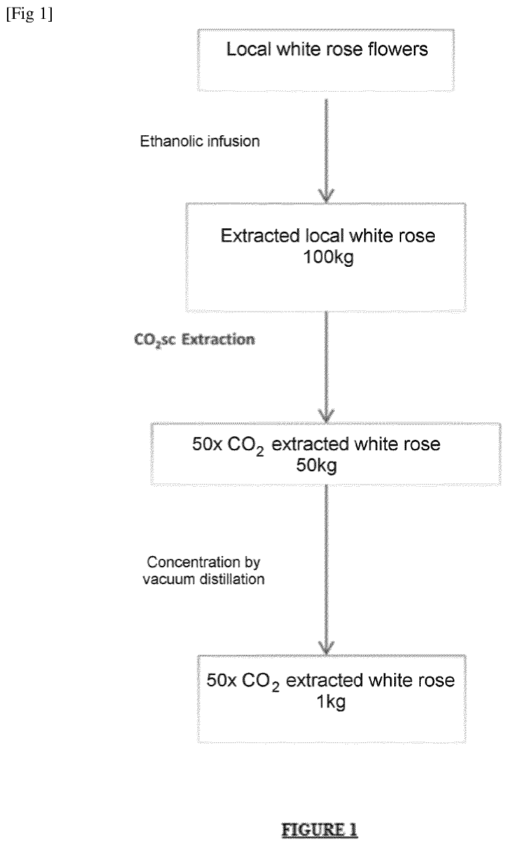

a) rose flowers, flowering tops and/or leaves are picked;
b) said freshly picked flowers, flowering tops and/or leaves are infused in at least one bath comprising an ethanolic solvent, at a temperature of less than 50° C., for example at room temperature, so as to obtain an alcoholic mixture (in this case an ethanolic mixture);
c) said alcoholic mixture is filtered so as to collect an alcoholic floral infusion; and
d) an extraction with supercritical CO₂ (“CO₂sc extraction” in FIG. 1) of the alcoholic floral infusion is performed so as to obtain said extract, at 45° C. and at a pressure of 150 bar; and
e) said extract is concentrated under a moderate vacuum (100 to 500 mbar), at a temperature not exceeding 60° C.

### Example 2

Migration and Re-Epithelialization Test on Keratinocytes

### A. Materials and Methods

### A1. Culture and Treatment of Keratinocytes

Keratinocytes were plated in a culture medium in a 96-well plate, and then devoted to migration analysis (ref. Platypus Oris™ Collagen I Coated Plate). In this plate, the wells were saturated with a collagen solution and a cover was placed in the center of each well, preventing the adhesion of the cells in this area, thus forming an artificial wound (migration area). After adhesion of the cells, the covers were discarded and the cells were labeled with calcein-AM. After incubation for 30 minutes, images were taken (T0) and the medium was then replaced with an assay medium containing or not containing (control) the test extract or the reference (EGF). The cells were incubated and image analyses were performed kinetically at the times 14 hours (T14), 18 hours (T18) and 24 hours (T24). Before the image taken at the time T14, the cells were again labeled with calcein-AM and incubated for 30 minutes. All the experimental conditions were performed in n=3.

### A2. Migration Analysis

The migration area of the cells was monitored after 0, 14, 18 and 24 hours of incubation with a high-resolution imaging system, INCell Analyzer™ 2200 automated microscope (GE Healthcare) (×4 lens) and the surface area of artificial wound was analyzed with the software Image J. The surface area of the artificial wound (central area without cells) was measured at the time T0, and after 14, 18 and 24 hours of incubation. In order to monitor and quantify the covering of the wound, the measurements of the wounds after 14, 18 and 24 hours of incubation were linked to the initial surface area measured at T0. The effect of the compounds on the migration was compared to the untreated control. The results are shown in tables 1 to 3 below.

### B. Results

Under the control condition, the migration of the normal human epidermal keratinocytes (NHEK) was moderate, with an average degree of covering of the surface area of wound of 41% after 14 hours of incubation. Within the next hours, the migration of NHEK increased to reach an average degree of covering of 55% after 24 hours of incubation.

Under the experimental conditions of this study, the supercritical CO₂ extract (CO₂sc extract) of rose according to the invention induces stimulation of the migration of normal human keratinocytes in a system which makes it possible to mimic an area of a homogeneous and artificial wound. The results obtained with the extract are, surprisingly, of the same amplitude as those obtained with the positive control (EGF). A 50% acceleration of the migration compared with the positive control is observed within the first hours of the migration.

TABLE 1

| | T0 | T14 | | |
|---|---|---|---|---|
| | Initial | Migration | | |
| | surface | area (% of | Mean | % |
| Treatment | area (mm²) | covering) | (%) | control |
| Control | 3.41 | 38 | 41 | 100 |
| | 3.16 | 41 | | |
| | 3.15 | 46 | | |
| EGF | 3.17 | 63 | 64 | 155(***) |
| (10 | 3.22 | 66 | | |
| ng/ml) | 3.07 | 63 | | |
| Extract | 3.06 | 63 | 64 | 154(**) |
| 0.00033% | 3.01 | 67 | | |
| | 2.98 | 62 | | |
| Extract | 3.22 | 62 | 64 | 156(**) |
| 0.001% | 2.81 | 68 | | |
| | 2.99 | 63 | | |
| Extract | 2.91 | 63 | 59 | 143(**) |
| 0.003% | 3.11 | 57 | | |
| | 2.87 | 57 | | |

Effect of an Extract on the Migration of Keratinocytes as a Function of Time (T0, T14)

TABLE 2

| | T0 | T18 | | |
|---|---|---|---|---|
| | Initial surface | Migration are | | |
| Treatment | area (mm²) | (% of covering) | Mean (%) | % control |
| Control | 3.41 | 44 | 47 | 100 |
| | 3.16 | 47 | | |
| | 3.15 | 51 | | |
| EGF | 3.17 | 70 | 72 | 152 (***) |
| (10 ng/ml) | 3.22 | 73 | | |
| | 3.07 | 72 | | |
| Extract | 3.06 | 68 | 71 | 149 (***) |
| 0.00033% | 3.01 | 74 | | |
| | 2.98 | 69 | | |
| Extract | 3.22 | 70 | 72 | 152 (***) |
| 0.001% | 2.81 | 75 | | |
| | 2.99 | 70 | | |
| Extract | 2.91 | 71 | 66 | 140 (**) |
| 0.003% | 3.11 | 65 | | |
| | 2.87 | 63 | | |

### Effect of an Extract on the Migration of Keratinocytes as a Function of Time (T0, T18)

TABLE 3

| | T0 | T24 | | |
|---|---|---|---|---|
| | Initial surface | Migration area | | |
| Treatment | area (mm²) | (% of covering) | Mean (%) | % control |
| Control | 3.41 | 53 | 55 | 100 |
| | 3.16 | 54 | | |
| | 3.15 | 58 | | |
| EGF | 3.17 | 83 | 85 | 154 (***) |
| (10 ng/ml) | 3.22 | 85 | | |
| | 3.07 | 87 | | |
| Extract | 3.06 | 80 | 84 | 152 (***) |
| 0.00033% | 3.01 | 89 | | |
| | 2.98 | 82 | | |
| Extract | 3.22 | 72 | 81 | 148 (***) |
| 0.001% | 2.81 | 89 | | |
| | 2.99 | 83 | | |
| Extract | 2.91 | 82 | 79 | 144 (***) |
| 0.003% | 3.11 | 79 | | |
| | 2.87 | 77 | | |

### Effect of an Extract on the Migration of Keratinocytes as a Function of Time (T0, T24)

For tables 1 to 3 above, the statistical significance threshold is:

### *: 0.01 to 0.05. Significant

**: 0.001 to 0.01. Very significant
***: <0.001. Extremely significant

### Example 3

An antiwrinkle emulsion having the following composition was prepared:

| | |
|---|---|
| Oily phase | 9% |
| Surfactants | 2% |
| CO₂ Extract of Meichibon × Delgramaue Rose | 0.001% |
| Aqueous phase | % |
| Polymer | 40% |
| Preserving agents | 0.50% |
| Alcohol | 3% |
| Water | qs |

The percentage values indicated correspond to mass percentages by weight relative to the total weight of the composition.

### Example 4

Comparative Study with a Second Hybrid Rosebush Extract

In the present study, an extract of hybrid rosebush Meichibon×Delgramaue, obtained by extraction with supercritical CO₂, according to the invention, is compared to a propylene glycol/water extract of another hybrid rosebush Rosa×Centifolia.

### A. Materials and Methods

### A1. The Tested Extracts

The supercritical CO₂ extract is obtained as already detailed in Example 1.

The propylene glycol/water extract of Rosa Centifolia Flower Extract (INCI: Propylene Glycol (and) Water (and) Rosa Centifolia Flower Extract) which is used throughout this example is commercializd by GATTEFOSSE SAS (36, chemin de Genas—BP 603-F-69804 Saint-Priest Cedex—France). Its characteristics are as follows:

TABLE 4

| | |
|---|---|
| Color (Gardner scale) | 6.0 to 9.0 |
| Density at 20° C. (D20/4) | 1.030 to 1.050 |
| Refraction Index at 20° C. | 1.383 to 1.396 |
| pH (pure product) | 4.5 to 6.0 |
| Dry extract | 0.2 to 1.5 g per 100 g |
| Heavy metal (Pb) | <20 ppm |
| Arsenic | <1 ppm |
| Total aerobic germs | <100/g |

### A2. Other Reagents

The tested keratinocytes are NHEK cells (Bioalternatives K593; 3^{rd} passage), cultivated at 37° C. under 5% CO₂. The cell culture medium is a keratinocyte-SFM medium supplemented with Epidermal Growth Factor (EGF) at 0.25 ng/mL, pituitary extract (EP) at 25 μg/mL and gentamycine at 25 μg/mL. Trial medium is the same as above, but without EGF nor EP.

### A3. Migration Analysis

The protocol is as defined in Example 1.

The percentage of recovery (%) is defined as: 100−[(wound recovery)/(wound surface at T0)*100]. Comparison between groups is achieved with bilateral Student's t-test (unpaired). A p value above 0.05 is considered as statistically not relevant. A p value at or below 0.05 is considered as statistically relevant and marked as (*). A p value at or below 0.01 is considered as statistically very relevant and marked as (**). A p value at or below 0.001 is considered as statistically extremely relevant and marked as (***).

A4. Preliminary Test for Cytotoxicity

The tested cells correspond to NHEK cells in trial medium, incubated for 24 hours. The cells are evaluated on, both, a MTT assay (tetrazolium salt) and a morphological study on the microscope.

### B. Results

Under the control condition, the migration of the normal human epidermal keratinocytes (NHEK) was moderate, with an average degree of covering of the surface area of wound of 30% after 14 hours of incubation. Within the next hours, the migration of NHEK increased to reach an average degree of covering of 37% after 24 hours of incubation. EGF at 10 ng/mL stimulates significantly the migration of NHEK and this effect is observed after 14 h, 18 h, and 24 h of incubation (respectively 221%, 223%, 208% when compared to the untreated sample). This result was expects and validates the study.

Then, the extract of hybrid rosebush Meichibon×Delgramaue (according to the invention) is tested at 0.01% and 0.03%. It is found that the extract stimulates significantly and in a concentration-dependent manner the migration of keratinocytes after 14 hours of incubation, respectively 141% and 160% when compared to the control condition. It is found that, under those conditions, the stimulating effect is of a similar amplitude for all the incubation conditions (14 h, 18 h, and 24 hours). The values

The detailed results are provided in Table 5 herebelow (corresponding to 14 hours of incubation).

Comparatively, the propylene glycol/water extract of hybrid rosebush Rosa×Centifolia is tested at 0.366% and 1.1%. When compared to the control condition, it is found that this extract inhibits significantly and in a concentration-dependent manner, the migration of keratinocytes after 14 hours of incubation (respectively 50% and 24% of the control condition). It is also found that, under those conditions, the inhibitory effect is of a similar amplitude for all the incubation conditions (14 h, 18 h, and 24 hours).

Accordingly, it is found that the hybrid rose extract according to the invention has a stimulating effect in vitro on the migration of keratinocytes, whereas another hybrid rose extract not belonging to the invention has an inhibitory effect.

TABLE 5

| | | | | Mean % | % of | |
|---|---|---|---|---|---|---|
| | | Wound surface | Wound surface | of | the | P |
| | Concent. | at T0 (mm²) | after 14 h (mm²) | migration | control | value |
| Control | — | 3.32/3.37/3.41 | 2.32/2.41/2.36 | 29.8 | 100 | — |
| EGF | 10 ng/ml | 3.18/3.18/3.31 | 1.07/1.08/1.13 | 66.1 | 221 | *** |
| (I) | 0.01% | 3.14/3.30/3.28 | 1.86/1.85/1.93 | 42 | 141 | *** |
| (I) | 0.03% | 3.11/3.17/3.36 | 1.42/1.57/2.08 | 47.6 | 160 | * |
| Rosa | 0.366% | 3.29/3.10/3.28 | 2.85/2.54/2.82 | 15.0 | 50 | *** |
| Rosa | 1.1% | 3.36/3.17/3.12 | 3.08/2.93/2.93 | 7.3 | 24 | *** |

The extract marked with (I) corresponds to the extract of hybrid rosebush Meichibon× Delgramaue, according to the invention.

The extract marked as “Rosa” corresponds to the propylene glycol/water extract of hybrid rosebush Rosa×Centifolia Wound surface values are calculated as a mean over three images, each.

The percentage values indicated correspond to mass percentages by weight relative to the total weight of the composition.

1. A rosebush extract, characterized in that said rosebush is a hybrid obtained by crossing the varieties Meichibon×Delgramaue.

2. The rosebush extract as claimed in claim 1, characterized in that said rosebush extract is obtained from flowers, flowering tops, and/or leaves of said rosebush.

3. The rosebush extract as claimed in claim 1, characterized in that said extract obtained by extraction with supercritical CO₂ of an alcoholic mixture of all or part of said rosebush.

4. The rosebush extract as claimed in claim 3, characterized in that said alcoholic mixture is obtained after infusion of all or part of said rosebush in at least one bath comprising an alcoholic solvent, at a temperature of less than 50° C., so as to obtain an alcoholic mixture.

5. A composition comprising a rosebush extract as claimed in claim 1.

6. A cosmetic use of an extract of a hybrid rosebush obtained by crossing the varieties Meichibon×Delgramaue, or of a composition comprising said extract, for caring for keratin materials.

7. The cosmetic use as claimed in claim 6, for treating and/or preventing cosmetic signs chosen from wrinkles, fine lines, wizened skin, loss of elasticity and/or tonicity and/or density of the skin, impairment of the radiance of the skin complexion, the papery appearance of the skin, slackening of the skin, the wizened appearance of the skin.

8. A cosmetic process for caring for keratin materials, comprising at least one step consisting in administering to an individual in need thereof, as an active agent, at least one rosebush extract, characterized in that said rosebush is a hybrid obtained by crossing the varieties Meichibon×Delgramaue.

9. The rosebush extract as claimed in claim 2, characterized in that said extract obtained by extraction with supercritical CO₂ of an alcoholic mixture of all or part of said rosebush.

10. The rosebush extract as claimed in claim 9, characterized in that said alcoholic mixture is obtained after infusion of all or part of said rosebush in at least one bath comprising an alcoholic solvent, at a temperature of less than 50° C., so as to obtain an alcoholic mixture.
